(19) United States
(12) Patent Application Publication
(10) Pub. No.: US 2025/0332030 A1
(43) Pub. Date: Oct. 30, 2025

(54) LIQUID CRYSTAL TINTABLE GOGGLES

(71) Applicant: Bell Sports, Inc., IRVINE, CA (US)

(72) Inventors: David M. THOMPSON, Irvine, CA (US); Mark A. BUSSE, Irvine, CA (US)

(21) Appl. No.: 19/194,260

(22) Filed: Apr. 30, 2025

Related U.S. Application Data
(60) Provisional application No. 63/640,305, filed on Apr. 30, 2024.

(51) Int. Cl. A61F 9/02; G02C 7/10

(52) U.S. Cl. CPC A61F 9/023; A61F 9/029; G02C 7/101

(57) ABSTRACT

A goggles is provided. The goggles can include a lens supported by a frame having a first exterior surface and a second exterior surface. An electronics housing can form at least a portion of the first exterior surface. A control interface can extend from the electronics housing to the second exterior surface, and can couple to the electronics housing to activate electronics disposed in the electronics housing to thereby transition the lens between the clear state and the tinted state.

### RELATED APPLICATION DATA

This application claims priority to U.S. Provisional Application No. 63/640,305, filed Apr. 30, 2024, the disclosure of which is hereby incorporated by reference in its entirety.

### FIELD

The present invention relates to goggles, and particularly ski goggles having a liquid crystal display.

### BACKGROUND

Goggles, such as for sporting activities, can be used in outdoor environments with varying conditions. For example, the daytime can have sunlit and bright conditions, while the evening and night can have overcast and dark conditions. Goggles that adapt to changing conditions can be advantageous and desirable for sporting activity participants. Liquid crystal displays can be used in goggles to vary the tint of goggles, allowing participants to adjust tint as desired. Participants can, for example, desire a darker tint during sunlit and bright conditions and a lighter tint in overcast and dark conditions. Switching between various tints can require a user action. In harsher outdoor conditions having lower temperatures or precipitation, such as the conditions experienced during skiing, it can be difficult for participants to adjust the tint of goggles. Accordingly, a need exists for improved systems and methods for adjusting a tint in goggles.

### SUMMARY

A goggles is provided. The goggles can include a frame including a first frame portion disposed on a first side of the goggles and a second frame portion disposed on a second side of the goggles opposite the first side. The goggles can also include a lens disposed between the first frame portion and the second frame portion. The goggles can also include an electronics housing forming at least a portion of a first exterior surface of the frame, the electronics housing including electronics configured to shift the lens between a clear state and a tinted state. The goggles can also include a control interface extending from at least a portion of a second exterior surface of the frame opposing the first portion of the exterior surface of the frame. The control interface can be configured to activate the electronics to thereby transition the lens between the clear state and the tinted state.

In some embodiments, the first frame portion includes the electronics housing and the control interface.

In some embodiments, the electronics include a printed circuit board (PCB). In some embodiments, the electronics further include a light pipe aligned with a light-emitting diode on the PCB.

In some embodiments, the second frame portion is void of electronics.

In some embodiments, the second frame portion includes a battery coupled to the electronics housing. In some embodiments, the first frame portion includes the electronics housing.

In some embodiments, the control interface extends through the frame from the second exterior surface of the frame to a PCB disposed in the electronics housing.

In some embodiments, the control interface extends through the lens.

In some embodiments, the lens includes a polycarbonate.

In some embodiments, the lens includes an outer portion and an inner portion coupled to the outer portion, the inner portion including a liquid crystal display (LCD) configured to shift the lens between the clear state and the tinted state.

A frame portion of a goggles is provided. The frame portion can include an electronics housing forming first portion of an exterior surface of the frame portion. The electronics housing can include electronics configured to shift a lens of the goggles coupled to the frame portion between a clear state and a tinted state, the lens being flush with the frame portion. The frame portion can also include a control interface forming a second portion of the exterior surface of the frame portion opposing the first portion. The control interface can be configured to activate the electronics to thereby transition the lens between the clear state and the tinted state.

In some embodiments, the control interface extends through the lens.

In some embodiments, the electronics housing is coupled to a battery.

In some embodiments, the control interface includes a button disposed in a button tray, the button tray coupled to the electronics housing.

A goggles is provided. The goggles can include a frame. The goggles can also include an electronics housing forming at least a portion of a first exterior surface of the frame. The electronics housing can include electronics configured to shift the lens between a clear state and a tinted state. The goggles can also include a control interface extending from the electronics housing to at least a portion of a second exterior surface of the frame opposing the first exterior surface of the frame. The control interface can be configured to activate the electronics to thereby transition the lens between the clear state and the tinted state.

In some embodiments, the electronics include a PCB.

In some embodiments, the electronics further include a light pipe aligned with a light-emitting diode on the PCB.

In some embodiments, the goggles can also include a battery coupled to the electronics housing; and a lens disposed between the battery and the electronics housing.

In some embodiments, the control interface includes a button disposed in a button tray, the button tray coupled to the electronics housing.

In some embodiments, the button is configured to extend from the second exterior surface of the frame, the button being coupled to the electronics such that the electronics are activatable from the second exterior surface.

### BRIEF DESCRIPTION OF THE DRAWINGS

The disclosed embodiments will hereinafter be described in conjunction with the appended drawings, provided to illustrate and not to limit the disclosed embodiments, wherein like designations denote like elements.

FIG. 1 shows an assembly view of goggles according to various embodiments.

FIG. 2 shows a perspective view of the goggles of FIG. 1.

FIG. 3 shows a top view of the goggles of FIG. 1.

FIG. 4 shows a rear view of the goggles of FIG. 1.

FIG. 5 shows an assembly view of a first frame portion of the goggles of FIG. 1.

FIG. 6 shows a rear view of the goggles of FIG. 1.

FIG. 7 shows a rear view of the goggles of FIG. 1.

FIG. 8 shows a side view of the goggles of FIG. 1.

FIG. 9 shows an assembly view of a lens of the goggles of FIG. 1.

FIG. 10 shows a front view of the lens of the goggles of FIG. 1.

FIG. 11 shows a front view of the lens of the goggles of FIG. 1.

### DETAILED DESCRIPTION

Various embodiments of the novel systems, apparatuses, and methods disclosed herein are described more fully hereinafter with reference to the accompanying drawings. This disclosure can, however, be embodied in many different forms and should not be construed as limited to any specific structure or function presented throughout this disclosure. Rather, these embodiments are provided so that this disclosure will be thorough and will fully convey the scope of the disclosure to those skilled in the art. Based on the teachings herein, one skilled in the art would appreciate that the scope of the disclosure can be intended to cover any embodiment of the novel systems, apparatuses, and methods disclosed herein, whether implemented independently of, or combined with, any other embodiment of the disclosure. For example, an apparatus can be implemented or a method can be practiced using any number or combination of the embodiments set forth herein. In addition, the scope of the disclosure can be intended to cover such an apparatus or method that can be practiced using other structure, functionality, or structure and functionality in addition to or other than the various embodiments of the disclosure set forth herein. It should be understood that any embodiment disclosed herein can be implemented by one or more elements of a claim.

Although particular embodiments are described herein, many variations and permutations of these embodiments fall within the scope of the disclosure. Although some benefits and advantages of the preferred embodiments are mentioned, the scope of the disclosure can be not intended to be limited to particular benefits, uses, and/or objectives. The detailed description and drawings are merely illustrative of the disclosure rather than limiting, the scope of the disclosure being defined by the appended claims and equivalents thereof.

It will be recognized that while certain embodiments of the disclosure are described in terms of a specific sequence of steps of a method, these descriptions are only illustrative of the broader methods of the disclosure, and can be modified as required by the particular application. Certain steps can be rendered unnecessary or optional under certain circumstances. Additionally, certain steps or functionality can be added to the disclosed embodiments, or the order of performance of two or more steps permuted. All such variations are considered to be encompassed within the disclosure disclosed and claimed herein.

While the disclosure has been illustrated and described in detail in the drawings and foregoing description, such illustration and description are to be considered illustrative or exemplary and not restrictive. The disclosure is not limited to the disclosed embodiments. Variations to the disclosed embodiments and/or implementations can be understood and effected by those skilled in the art in practicing the claimed disclosure, from a study of the drawings, the disclosure and the appended claims.

Goggles can be used in various sporting activities, such as for skiing. These activities may be conducted outdoors in which lighting conditions change as the day progresses. For example, the daytime can have sunlit and bright conditions, while the evening and night can have overcast and dark conditions. Goggles can be configured to adapt to changing lighting conditions. Some goggles can include a lens having a liquid crystal display that adjust a tint of the lens, which can block at least a portion of light from crossing the lens to the user's eyes, providing the user with better visibility. In sunlit and bright conditions, the user may desire a tinted lens to block sunlight. In addition to allowing the user to have better visibility, blocking sunlight can protect the user's eyes from ultraviolet radiation. In overcast or darker conditions, the user may desire a clear lens without tint to allow any light to pass through the lens to aid in visibility.

Goggles can include one or more user actuators to adjust a tint. These user actuators can be located on a surface of the goggles that is accessible to the wearer when the goggles are in use. Some user actuators can be difficult to actuate, particularly in outdoor conditions with harsher weather, such as snow and wind. The user actuators may be inconveniently located on the goggles, or they may require significant interaction from the user to adjust a tint. Users require actuators that are practically located and accessible. In addition, users desire goggles that are aesthetically pleasing. Accordingly, user actuators to adjust a tint of goggles must also be discrete to maintain a desirable appearance of the goggles.

Systems and methods described herein provide goggles that transition a lens of the goggles between multiple states representing the presence or absence of a tint. The goggles can include a frame having one or more frame portions to support a lens. At least one frame portion can include electronics having a printed circuit board and a light pipe for adjusting a tint of the lens. At least one frame portion can include a power source for the electronics. The frame can also include a control interface to activate the electronics to thereby transition the lens between a clear state and a tinted state.

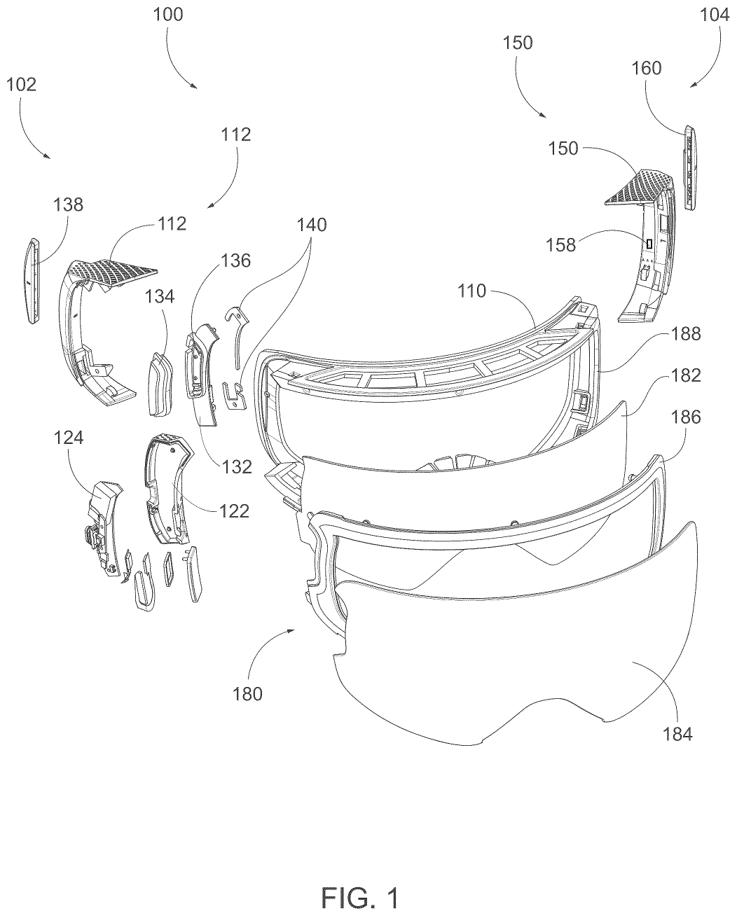

Systems and methods will now be described with reference to the figures. FIG. 1 illustrates goggles 100, according to an exemplary embodiment. Goggles 100 can include a frame 110 having a carrier 188. In addition, goggles 100 can include a lens 180 having an inner lens 182, an outer lens 184, and a gasket 186 therebetween. Along a first side 102 of goggles 100, frame 110 can include a first frame portion 112. Along a second side 104 of goggles 100, frame 110 can include a second frame portion 150. Goggles 100 can be wearable by a wearer, such as via straps. Goggles 100 can include a first strap end 138 disposed on first side 102 and a second strap end 160 disposed on second side 104 to receive straps that can wrap around the wearer's head or connect to a helmet.

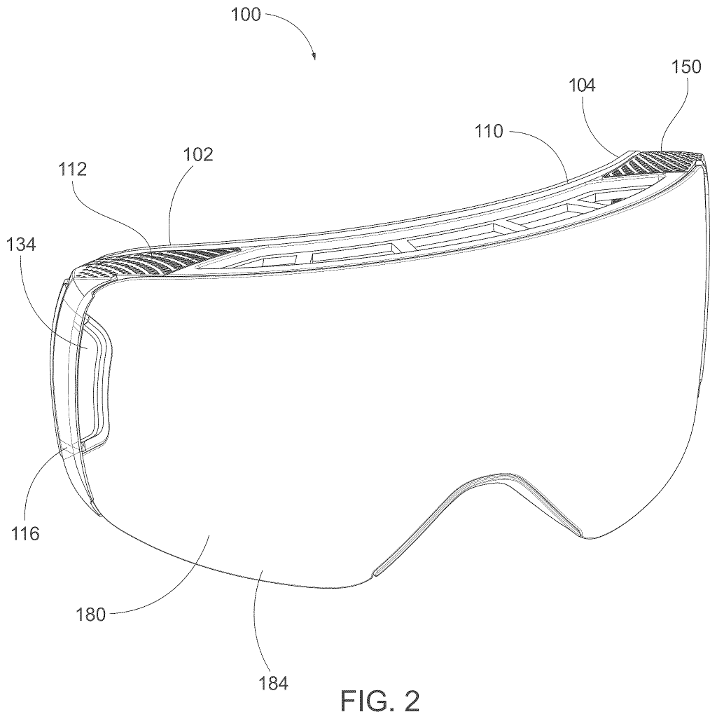

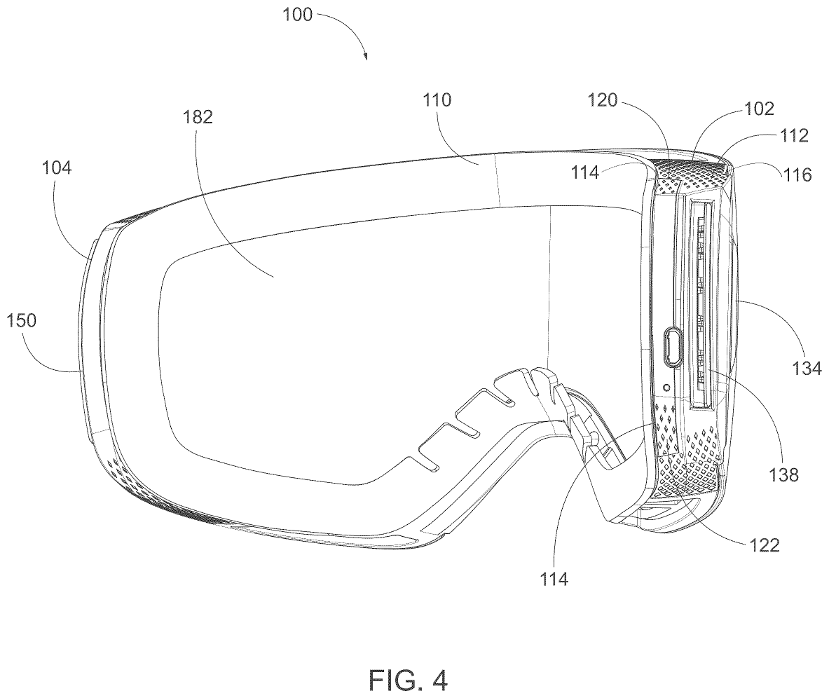

Goggles 100 in an assembled state is shown in FIGS. 2-4, according to an exemplary embodiment. As shown in FIG. 2, for example, goggles 100 can include frame 110 having first frame portion 112 disposed on first side 102 of goggles 100, and second frame portion 150 disposed along second side 104 opposite first side 102. Lens 180 can be disposed between first frame portion 112 and second frame portion 150.

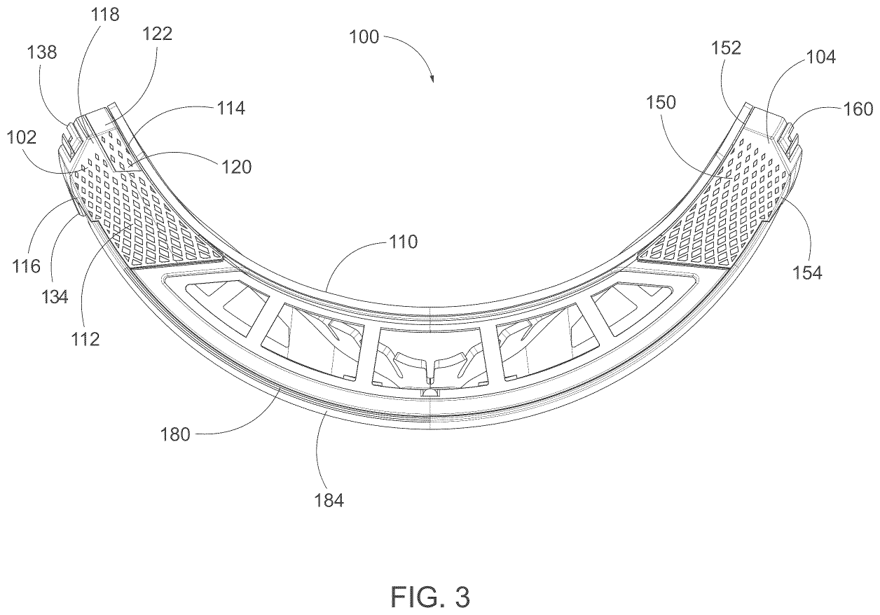

With reference to FIG. 3, frame 110 can include a first exterior surface 114 and a second exterior surface 116 opposing first exterior surface 114. First frame portion 112 and second frame portion 150 forming frame 110 can each include first exterior surface 114 and second exterior surface 116. First exterior surface 114 and second exterior surface 116 can form opposing exterior surfaces of frame 110. Lens 180 can be disposed adjacent second exterior surface 116, as shown.

Frame 110, such as first frame portion 112, or second frame portion 150, can also include an electronics housing 122. Electronics housing 122 can form first exterior surface 114. As shown in FIG. 3, electronics housing 122 can form at least a portion of first exterior surface 114. In this way, at least first frame portion 112 can form second exterior surface 116, and both first frame portion 112 and electronics housing 122 can form first exterior surface 114.

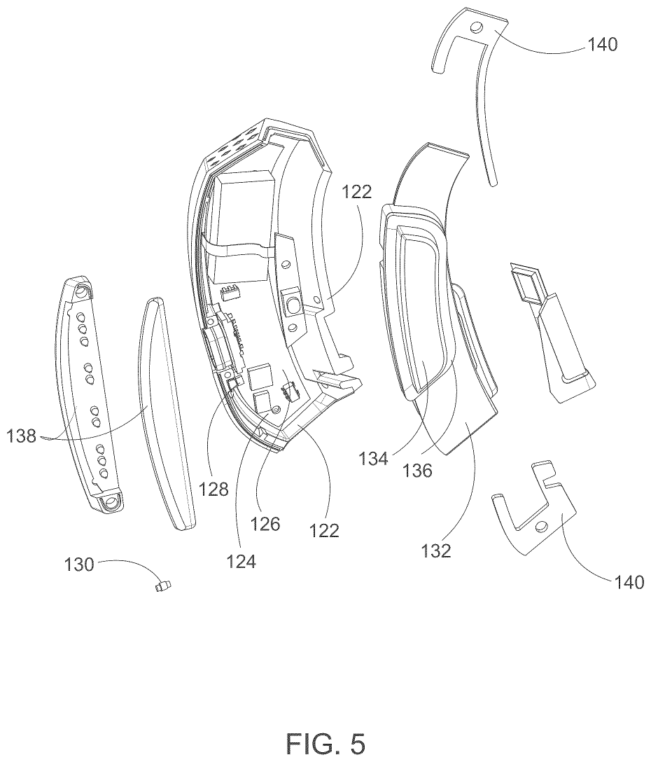

As shown with reference to FIG. 5, according to an exemplary embodiment, electronics housing 122 can be a housing that includes electronics 124. Electronics housing 122 can shift lens 180 (FIG. 2) between a clear state, in which lens 180 can include little or no tint to allow a majority or all of light to pass through, and a tinted state, in which lens 180 can include tint to block light from passing through. Electronics 124 can include a printed circuit board (PCB) 126. Electronics 124 can also include a light pipe 130 aligned with a light-emitting diode (LED) 128 on PCB 126.

A control interface 132 can activate electronics 124 to thereby transition lens 180 between the clear state and the tinted state. Referring to FIGS. 2 and 5, control interface 132 can be disposed on any portion of goggles 100. For example, frame 110, such as first frame portion 112, or second frame portion 150, can include control interface 132. In an example, first frame portion 112 can include electronics housing 122 and control interface 132.

Control interface 132 can include one or more user actuators to activate electronics 124 and adjust a tint and shift between the clear state and the tinted state. For example, goggles 100 can include a button 134, which can be a user actuator. In other examples, the user actuator can include a toggle, a switch, a push button, a dial, or any other means for user actuation. Button 134 can be accessible to the wearer of goggles 100 when goggles 100 are worn, or otherwise in use. Referring to FIG. 5, control interface 132 can include a button 134. Button 134 can be disposed in a button tray 136, which can be coupled to electronics housing 122. Accordingly, a user actuating button 134 can actuate control interface 132 to activate electronics 124 disposed in electronics housing 122 and adjust a tint and shift between the clear state and the tinted state.

As goggles 100 can be used in outdoor conditions with harsher weather, such as snow and wind, button 134 can be practically located along goggles 100 to aid in accessibility, such as along an edge of goggles 100. Button 134 along an edge of goggles 100 can be seen in FIGS. 2-4, for example. Referring to FIGS. 2-4, to additionally achieve an aesthetically pleasing appearance, button 134 can be disposed on goggles 100 such that button 134 is less discernable or conspicuous in comparison to surrounding portions of goggles 100. Button 134 can be disposed on second exterior surface 116, for example, such that button 134 blends in with second exterior surface 116. Button 134 blending in such that button 134 is less discernable or conspicuous can mean that button 134 is a similar color or texture as second exterior surface 116. Additionally or alternatively, button 134 blending in such that button 134 is less discernable or conspicuous can mean that button 134 is small in size in comparison to second exterior surface 116. Additionally or alternatively, button 134 blending in such that button 134 is less discernable or conspicuous can mean that button 134 minimally protrudes from second exterior surface 116. As shown, button 134 can extend from second exterior surface 116 such that it does not protrude far beyond lens 180.

Button 134 can extend from second exterior surface 116 on first frame portion 112 or second frame portion 150 and couple to electronics 124 in first frame portion 112 or second frame portion 150 such that electronics 124 are activatable from second exterior surface 116. As discussed, first frame portion 112 can include electronics housing 122 and control interface 132. Accordingly, first frame portion 112 can include electronics 124. Second frame portion 150 can be void of electronics housing 122 and control interface 132 such that second frame portion 150 can be void of electronics 124. Alternatively, second frame portion 150 can include electronics housing 122 and control interface 132, while first frame portion 112 is void of electronics housing 122 and control interface 132, and therefore electronics 124.

First frame portion 112 or second frame portion 150 can additionally or alternatively house other components of goggles 100. For example, second frame portion 150, being void of electronics housing 122 and control interface 132 such that second frame portion 150 can be void of electronics 124, can include a battery 158, as shown in FIG. 1. Battery 158 can be coupled to electronics housing 122, which can be disposed in first frame portion 112 or second frame portion 150. In an example, second frame portion 150 can include battery 158 coupled to electronics housing 122, where first frame portion 112 can include electronics housing 122. In other words, battery 158 can be coupled to electronics housing 122, where lens 180 can be disposed between battery 158 and electronics housing 122.

Referring again to FIGS. 2-4, at least a portion of control interface 132 (FIG. 5) can extend through lens 180. In this way, button 134 disposed on control interface 132 can be accessible through lens 180 for user actuation. At least a portion of control interface 132 can form at least a portion of second exterior surface 116 such that the portion of control interface 132 can extend through lens 180, which can be disposed adjacent second exterior surface 116. In one example, lens 180 can include an opening through which the portion of control interface 132 can extend. In another example, lens 180 can be shaped such that control interface 132 can extend adjacent lens 180. In this way, lens 180 can be disposed adjacent first frame portion 112 and the portion of control interface 132, such as button 134, as shown in FIG. 3.

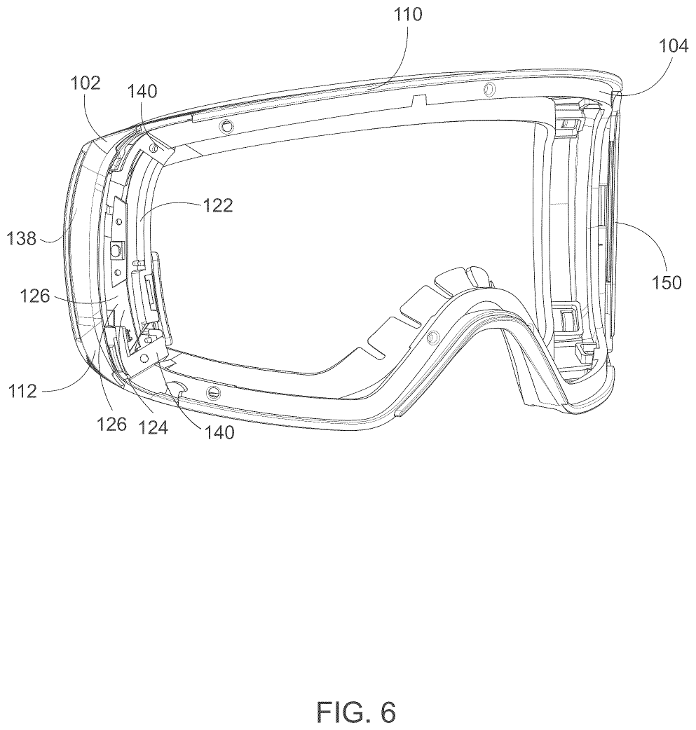

Electronics housing 122 can form a first portion of an exterior surface of frame 110, such as first frame portion 112 or second frame portion 150, electronics housing 122 including electronics 124 to shift lens 180 between a clear state and a tinted state, lens 180 being flush, or generally even with first frame portion 112 or second frame portion 150. In addition, control interface 136 (FIG. 5) can form a second portion of the exterior surface of frame 110 opposing the first portion, control interface 136 to activate electronics 124 to thereby transition the lens between the clear state and the tinted state. The first portion can be first exterior surface 114 and the second portion can be second exterior surface 116. To this end, control interface 136 (FIG. 5) can extend to second exterior surface 116 and from second exterior surface 116 to a portion of first frame portion 112 or second frame portion 150 intermediate to second exterior surface 116 and first exterior surface 114. As will be discussed next with reference to FIG. 6, electronics housing 122 can be disposed between control interface 136 and first exterior surface 114 such that control interface 136 can extend from second exterior surface 116 to a portion of first frame portion 112 or second frame portion 150 intermediate to second exterior surface 116 and first exterior surface 114.

FIG. 6 shows goggles 100 with electronics housing 122 disposed in first frame portion 112, according to an exemplary embodiment. In other embodiments, electronics housing 122 can be disposed in second frame portion 150. As discussed, control interface 136 (FIG. 5) can be coupled to electronics housing 122 such that control interface 136 can activate electronics 124 in electronics housing 122 and adjust a tint and shift between the clear state and the tinted state. Control interface 136 can couple to electronics housing 122 via tape 140, shown in FIG. 6. Control interface 136 is removed to make visible the coupling means and electronics housing 122. As control interface 136 is removed, it can be seen that control interface 136 can extend through frame 110, such as first frame portion 112 or second frame portion 150, such as first frame portion 112, from second exterior surface 116 to electronics housing 122 and components thereof. In other words, control interface 136 can extend from electronics housing 122 and components thereof to at least a portion of second exterior surface 116. In this way, control interface 136, and components thereof such as button 134, can extend through frame 110 from second exterior surface 116 to PCB 126 of electronics 124 disposed in electronics housing 122. Accordingly, control interface 136 can activate electronics 124 in electronics housing 122 and adjust a tint and shift between the clear state and the tinted state. In addition, control interface 136 can be accessible from an exterior of goggles 100.

Control interface 136 (FIG. 5) can extend to second exterior surface 116 and from second exterior surface 116 to a portion of first frame portion 112 or second frame portion 150 intermediate to second exterior surface 116 and first exterior surface 114, as control interface 136 can couple to electronics housing 122 disposed in first frame portion 112 or second frame portion 150. In other words, referring again to FIGS. 2-4, control interface 136 can form at least a portion of second exterior surface 116. In this way, first frame portion 112 and electronics housing 122 can form first exterior surface 114, and both first frame portion 112 and control interface 136 can form second exterior surface 116.

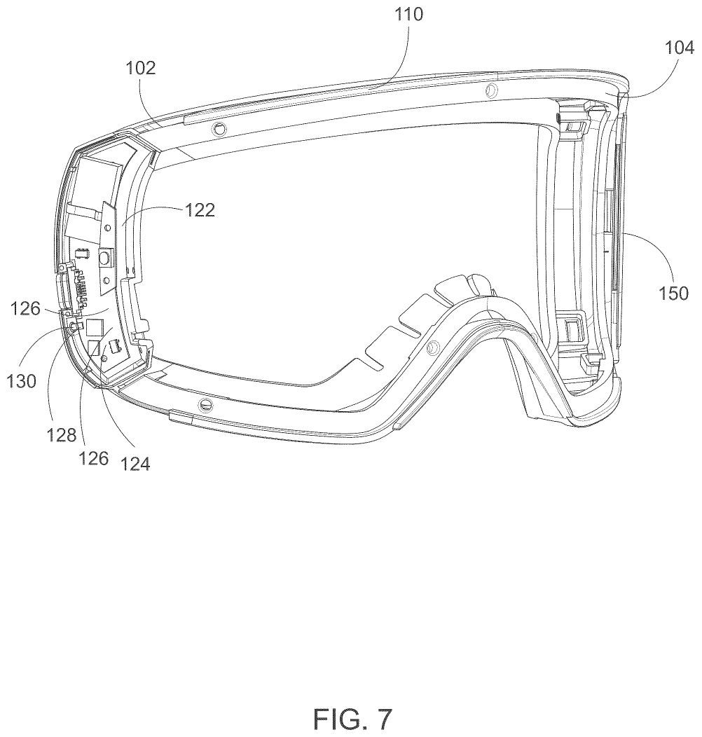

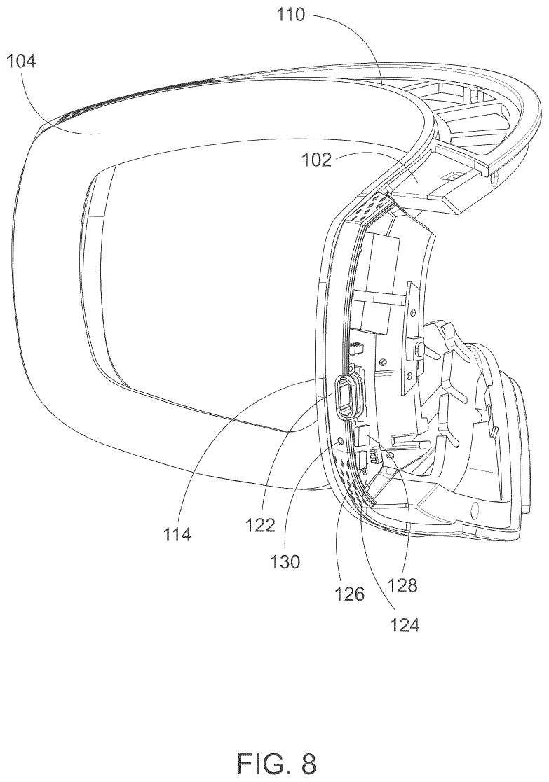

FIGS. 7-8 show goggles 100 with tape 140 removed, as well as first frame portion 112 (FIG. 6) and strap end 138 (FIG. 6). Accordingly, it can be seen that electronics housing 122 can extend from control interface 136 (FIG. 5) to first exterior surface 114.

Referring again to FIG. 3, for example, control interface 136 (FIG. 5), and components thereof such as button 134, extending from second exterior surface 116 can form second exterior surface 116, such as at least a portion of second exterior surface 116 opposing first exterior surface 114. Control interface 136 can be flush, or generally even, with first frame portion 112 or second frame portion 150, such as first frame portion 112, such as second exterior surface 116 of first frame portion 112. In this way, button 134 can minimally extend from first frame portion 112 to be less conspicuous. In addition, electronics housing 122 extending from first exterior surface 114 can form first exterior surface 114, such as at least a portion of first exterior surface 114. Electronics housing 122 can be flush, or generally even, with first frame portion 112 or second frame portion 150, such as first frame portion 112, such as first exterior surface 136 of first frame portion 112.

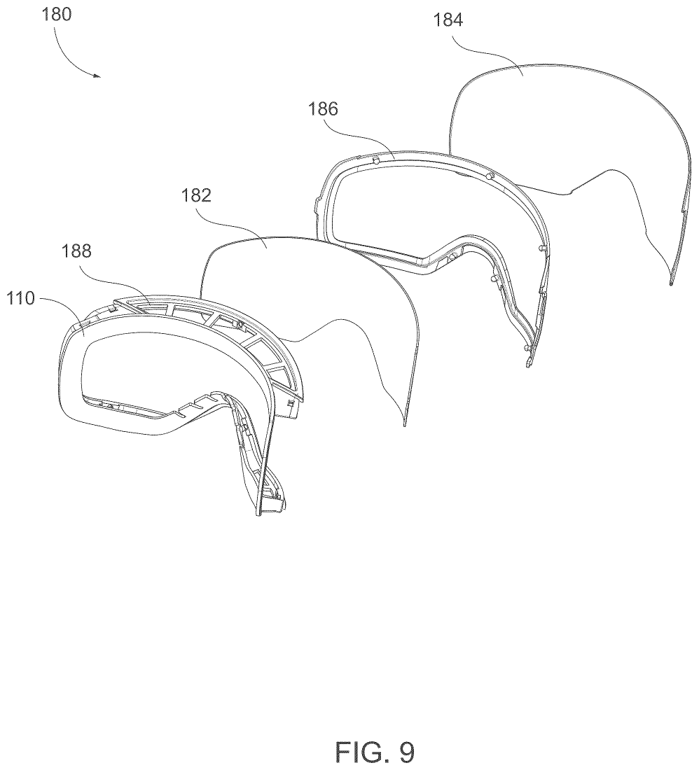

As discussed, goggles 100 can include lens 180, which can be seen in FIGS. 9-11, according to an exemplary embodiment. Lens 180 can be tintable such that a percentage of visible light transmission (VLT) can be allowed to pass through lens 180. For example, in the clear state described herein, lens 180 can allow between approximately 40 percent and approximately 100 percent of light to pass through lens 180, such as between approximately 45 percent and approximately 60 percent, such as approximately 50 percent. In the tinted state described herein, lens 180 can allow between approximately 0 percent and approximately 30 percent of light to pass through lens 180, such as between approximately 10 percent and approximately 25 percent, such as approximately 20 percent.

Lens 180 can include one or more lenses. At least one lens can include a polycarbonate. In addition, at least one lens being a liquid crystal display (LCD) to shift lens 180 between the clear state and the tinted state. As shown in FIG. 9, for example, lens 180 can include an outer portion 184, which can be an outer lens. Lens 180 can also include an inner portion 182, which can be an inner lens. Outer portion 184 can be coupled to inner portion 182. A gasket 186 can be disposed between outer portion 184 and inner portion 182 to couple and create a seal between outer portion 184 and inner portion 182. Outer portion 184 can include a polycarbonate, such as injection polycarbonate. Inner portion 182 can be an LCD, such as an LCD that is laminated to the back of outer portion 184. Inner portion 182 being an LCD can change the tint of lens 180 and shift lens 180 between the clear state and the tinted state.

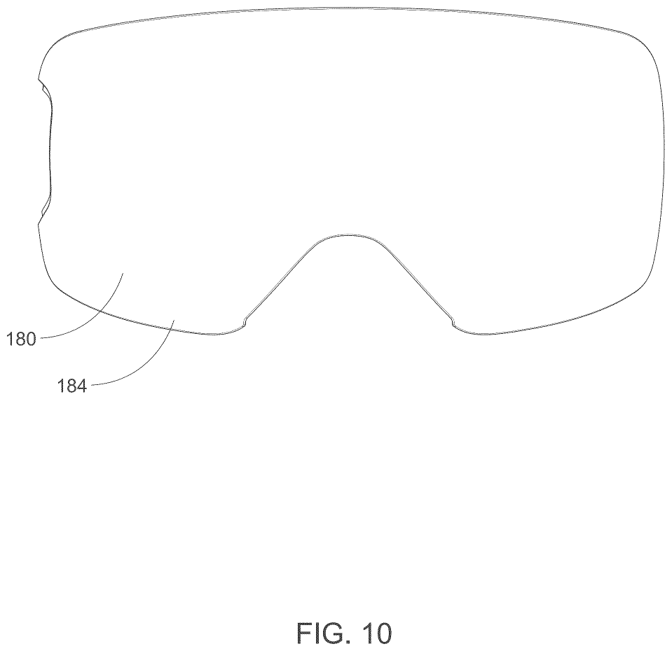

Lens 180 can include an opening to receive at least a portion of control interface 136 (FIG. 5), as discussed. Alternatively, lens 180 can include be shaped such that lens 180 is adjacent the portion of control interface 136. Lens 180 with a shape to accommodate control interface 136 is shown in FIG. 10, according to an exemplary embodiment.

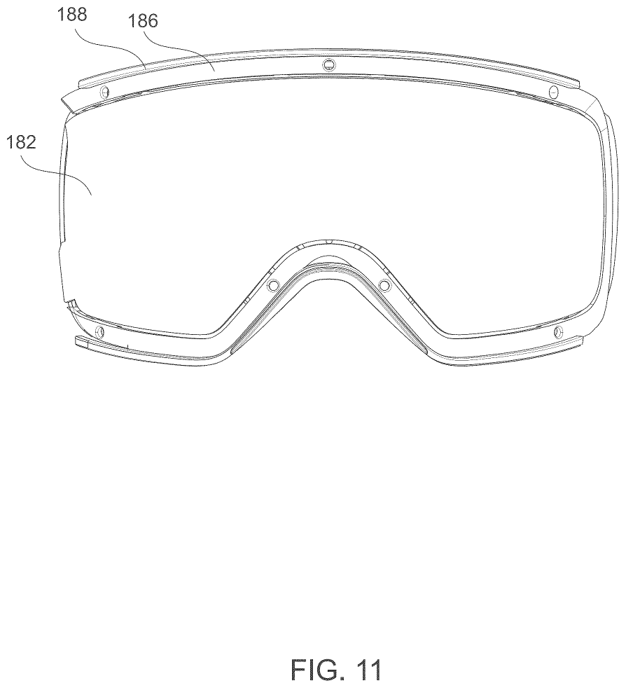

Lens 180 and components thereof can be secured to frame 188, shown with reference to FIG. 11, according to an exemplary embodiment. Referring to FIGS. 9 and 11, a carrier 186 can be a housing that can receive inner portion 182, gasket 186, and outer portion 182.

With reference to FIGS. 1-11, lens 190 can be secured to frame 188 by a friction fit, a magnetic attachment, or another fastening means. In an example, frame 188 can include one or more grooves which can receive lens 190 to retain lens 190 in frame 188. The grooves can receive a projection extending from lens 190, in an example. In another example, a retention mechanism can protrude from frame 188 and extend around lens 190 to retain lens 190. In another example, lens 190 can include apertures to receive protrusions from frame 188 to retain lens 190. Another example can include lens 190 and frame 188 being magnetically attached. Frame 188 can include a magnet that can attract a magnet of lens 190. In an example, frame 188 can include a recess having a magnet, and lens 190 can include a protrusion having a magnet that can attract the magnet of frame 188. In another example, frame can include a protrusion having a magnet, and lens 190 can include a protrusion having a magnet that can attract the magnet of frame 188. Magnetically attached lens 190 and frame 188 can particularly allow for lens 190 to easily and quickly be removed from frame 188, such as if another lens is desirable for use. However, it can be desirable to retain lens 190 in frame 188, as lens 190 can easily and quickly be adjusted between a clear state and a tinted state, reducing or negating the need for removing and replacing lens 190 with another lens.

It should be noted that the use of particular terminology when describing certain features or embodiments of the disclosure should not be taken to imply that the terminology is being re-defined herein to be restricted to include any specific characteristics of the features or embodiments of the disclosure with which that terminology is associated. Terms and phrases used in this application, and variations thereof, especially in the appended claims, unless otherwise expressly stated, should be construed as open-ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least”; the term “such as” should be interpreted as “such as, without limitation”; the term “includes” should be interpreted as “includes but is not limited to”; the term “example” is used to provide exemplary instances of the item in discussion, not an exhaustive or limiting list thereof, and should be interpreted as “example, but without limitation”; adjectives such as “known,” “normal,” “standard,” and terms of similar meaning should not be construed as limiting the item described to a given time period or to an item available as of a given time, but instead should be read to encompass known, normal, or standard technologies that can be available or known now or at any time in the future; and use of terms like “preferably,” “preferred,” “desired,” or “desirable,” and words of similar meaning should not be understood as implying that certain features are critical, essential, or even important to the structure or function of the present disclosure, but instead as merely intended to highlight alternative or additional features that can or cannot be utilized in a particular embodiment.

Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise. The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (e.g., measurement value) is close to a targeted value, where close can mean, for example, the result is within 80% of the value, within 90% of the value, within 95% of the value, or within 99% of the value. Also, as used herein “defined” or “determined” can include “predefined” or “predetermined” and/or otherwise determined values, conditions, thresholds, measurements, and the like.

What is claimed is:

1. A goggles, comprising:
a frame comprising a first frame portion disposed on a first side of the goggles and a second frame portion disposed on a second side of the goggles opposite the first side;
a lens disposed between the first frame portion and the second frame portion;
an electronics housing forming at least a portion of a first exterior surface of the frame, the electronics housing comprising electronics configured to shift the lens between a clear state and a tinted state; and
a control interface extending from at least a portion of a second exterior surface of the frame opposing the first portion of the exterior surface of the frame, the control interface being configured to activate the electronics to thereby transition the lens between the clear state and the tinted state.

2. The goggles of claim 1, wherein the first frame portion comprises the electronics housing and the control interface.

3. The goggles of claim 1, wherein the electronics comprise a printed circuit board (PCB).

4. The goggles of claim 3, wherein the electronics further comprise a light pipe aligned with a light-emitting diode on the PCB.

5. The goggles of claim 1, wherein the second frame portion is void of electronics.

6. The goggles of claim 1, wherein the second frame portion comprises a battery coupled to the electronics housing, and
wherein the first frame portion comprises the electronics housing.

7. The goggles of claim 1, wherein the control interface extends through the frame from the second exterior surface of the frame to a PCB disposed in the electronics housing.

8. The goggles of claim 1, wherein the control interface extends through the lens.

9. The goggles of claim 1, wherein the lens comprises a polycarbonate.

10. The goggles of claim 1, wherein the lens comprises an outer portion and an inner portion coupled to the outer portion, the inner portion comprising a liquid crystal display (LCD) configured to shift the lens between the clear state and the tinted state.

11. A frame portion of a goggles, comprising:
an electronics housing forming first portion of an exterior surface of the frame portion, the electronics housing comprising electronics configured to shift a lens of the goggles coupled to the frame portion between a clear state and a tinted state, the lens being flush with the frame portion; and
a control interface forming a second portion of the exterior surface of the frame portion opposing the first portion, the control interface being configured to activate the electronics to thereby transition the lens between the clear state and the tinted state.

12. The frame portion of claim 11, wherein the control interface extends through the lens.

13. The frame portion of claim 11, wherein the electronics housing is coupled to a battery.

14. The frame portion of claim 11, wherein the control interface comprises a button disposed in a button tray, the button tray coupled to the electronics housing.

15. A goggles, comprising:
a frame;
an electronics housing forming at least a portion of a first exterior surface of the frame, the electronics housing comprising electronics configured to shift the lens between a clear state and a tinted state; and
a control interface extending from the electronics housing to at least a portion of a second exterior surface of the frame opposing the first exterior surface of the frame, the control interface being configured to activate the electronics to thereby transition the lens between the clear state and the tinted state.

16. The goggles of claim 15, wherein the electronics comprise a PCB.

17. The goggles of claim 16, wherein the electronics further comprise a light pipe aligned with a light-emitting diode on the PCB.

18. The goggles of claim 15, further comprising:
a battery coupled to the electronics housing; and
a lens disposed between the battery and the electronics housing.

19. The goggles of claim 15, wherein the control interface comprises a button disposed in a button tray, the button tray coupled to the electronics housing.

20. The goggles of claim 19, wherein the button is configured to extend from the second exterior surface of the frame, the button being coupled to the electronics such that the electronics are activatable from the second exterior surface.
